(19) United States
(12) Patent Application Publication
(10) Pub. No.: US 2004/0229372 A1
(43) Pub. Date: Nov. 18, 2004

(54) Ozone indicator and method of measuring ozone concentration

(76) Inventors: Takeshi Omatsu, Kyotanabe-shi (JP); Satoshi Maruyama, Yao-shi (JP)

Correspondence Address: KNOBBE MARTENS OLSON & BEAR LLP, 2040 MAIN STREET, FOURTEENTH FLOOR, IRVINE, CA, 92614, US

(21) Appl. No.: 10/868,230

(22) Filed: Jun. 15, 2004

Related U.S. Application Data
(63) Continuation of application No. 09/871,752, filed on Jun. 1, 2001.

(30) Foreign Application Priority Data
Jun. 5, 2000 (JP) 2000-167653
Aug. 29, 2000 (JP) 2000-259716

(51) Int. Cl. G01N 33/00

(52) U.S. Cl. 436/135

(57) ABSTRACT

An ozone indicator comprising at least (1) a color-change layer comprised of an ozone sensitive ink and (2) an overcoat layer formed on part or the whole of the surface of said color-change layer, wherein the ozone sensitive ink comprises a color component which does not change color in an ozone atmosphere.

### CROSS-REFERENCE TO RELATED APPLICATION

This is a continuation application of U.S. patent application Ser. No. 09/871,752, filed Jun. 1, 2001, which claims priority to Japanese Patent Application Nos. 2000-167653, filed Jun. 5, 2000 and 2000-259716, filed Aug. 29, 2000. The disclosure of the United States Patent Application is herein incorporated by reference in its entirety.

### BACKGROUND OF THE INVENTION

1. Field of the Invention

The present invention relates to an ozone indicator and a method of measuring ozone concentration.

2. Description of the Related Art

Ozone, by virtue of its high germicidal and other characters, has been utilized for the sterilization and disinfection of foods and instruments, or for the sterilization, disinfection or deodorization of the atmosphere in confined spaces such as the operation rooms of hospitals. On the other hand, ozone is so toxic and deleterious to human health that there is naturally an allowable limit to its concentration. Meanwhile, in photochemical smog forecasts, the atmospheric oxidant level is an important parameter.

For the monitor of ozone concentration, therefore, a variety of methods have been developed for its detection. The principal ozone (oxidant) detection technology available so far takes advantage of the color change according to the reaction depicted in the following scheme (1).

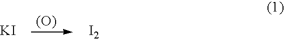

As specific detection methods utilizing the above principle, the optical method which comprises introducing an ozone-containing gas into a solution of potassium iodide and measuring the degree of resultant color change, which is proportional to the amount of liberated iodine, by means of a colorimeter and the expedient method utilizing a simple sensor tube are known.

However, these detection methods are invariably intended to detect comparatively low concentrations of ozone and not suitable for detecting high concentrations of ozone. Thus, when an attempt is made to detect a high level of ozone by these methods, the color change occurs in an instant so that even though the presence or absence of ozone can be detected, the ozone concentration and CT (product of ozone concentration by exposure time) value, among other parameters, cannot be quantified.

### SUMMARY OF THE INVENTION

Therefore, a prime object of the present invention is to provide an ozone indicator with which the ozone concentration, for instance, can be found even in an atmosphere containing an unusually high concentration of ozone.

The inventor did much research in view of the above disadvantage of the prior art and found that the above object can be accomplished by means of an ozone indicator having the herein-defined construction. The present invention has been developed on the basis of the above finding.

### BRIEF DESCRIPTION OF THE DRAWINGS

FIG. 1 is a diagram showing the results obtained in Test Example 1.

Graphs 1 and 4 show CT dependency data; graphs 2 and 5 show humidity dependency data; and graphs 3 and 6 show temperature dependency data.

### DETAILED DESCRIPTION OF THE PREFERRED EMBODIMENTS

The present invention relates to the following ozone indicators and methods of measuring ozone concentration.

1. An ozone indicator comprising at least (1) a color-change layer comprised of an ozone sensitive ink and (2) an overcoat layer formed on part or the whole of the surface of said color-change layer.

2. The ozone indicator according to the above paragraph 1 wherein the ozone sensitive ink contains an anthraquinone dye having at least one amino group species selected from the class consisting of primary and secondary amino groups.

3. The ozone indicator according to the above paragraph 2 wherein the ozone sensitive ink further contains a cationic surfactant of the quaternary ammonium salt type.

4. The ozone indicator according to the above paragraph 3 wherein the cationic surfactant of the quaternary ammonium salt type is an alkyltrimethylammonium salt.

5. The ozone indicator according to the above paragraph 2 wherein the ozone sensitive ink further contains an extender.

6. The ozone indicator according to claim 2 wherein the ozone sensitive ink further contains a resinous binder.

7. The ozone indicator according to the above paragraph 2 wherein the ozone sensitive ink further contains a color component which does not change color in an ozone atmosphere.

8. The ozone indicator according to the above paragraph 1 wherein the overcoat layer comprises a film-forming polymer.

9. The ozone indicator according to the above paragraph 8 wherein the film-forming polymer (resin) is at least one species of water-soluble polymer.

10. The ozone indicator according to the above paragraph 1 wherein the overcoat layer does not contain a coloring agent.

11. A method of measuring ozone concentration characterized by disposing the ozone indicator according to the above paragraph 1 in an ozone atmosphere and calculating a CT value from the color difference resulting from the change in color or the size of a color change zone of the color-change layer.

12. The measuring method according to the above paragraph 11 wherein the concentration of ozone in the ozone atmosphere is not less than 1000 ppm.

The ozone indicator of the invention is characterized in that it comprises at least (1) a color-change layer comprised of an ozone sensitive ink and (2) an overcoat layer formed on part or the whole of the surface of said color-change layer.

The above-mentioned color-change layer is a layer which changes color in the presence of ozone and is composed, for the most part, of an ozone sensitive ink. The “color change” in the context of the invention is a concept subsuming all events of change in an initial color (brightness value, hue, etc.), thus including the fading, decolorization and color development. The color-change layer may be formed on a substrate or, when the color-change layer is formable for itself (shape-retaining or self-standing), may be provided without the aid of a substrate.

When a substrate is used, the kind of substrate is not particularly restricted but includes metals, metal alloys, wood, paper, ceramics, glass, concrete, plastics, fibrous materials (non-woven cloth, woven cloth, and other fibrous sheets), etc. and composites thereof.

The ozone sensitive ink for forming the color-change layer is not particularly restricted insofar as it changes color on exposure to an ozone atmosphere (an ozone-containing atmosphere) and, as such, various known and/or commercial inks can be employed. For example, inks based on potassium iodide, triphenylmethane leuco dye, p-n-butoxyaniline, etc. can be utilized.

In the present invention, it is particularly preferable to use an ozone sensitive ink containing an anthraquinone dye having at least one amino group species selected from the class consisting of primary and secondary amino groups.

The anthraquinone dye for use in the invention is not particularly restricted insofar it has an anthraquinone nucleus as its fundamental skeletal structure and has at least one amino group species selected from among primary and secondary amino groups. The known disperse dyes in the anthraquinone series can also be employed. The amino group mentioned above may be present singly or in a plural number and, in the latter case, the amino groups may be the same or different.

Such anthraquinone dyes include 1,4-diaminoanthraquinone (C. I. Disperse Violet 1), 1-amino-4-hydroxy-2-methoxyanthraquinone (C. I. Disperse Red 4), 1-amino-4-methylaminoanthraquinone (C. I. Disperse Violet 4), 1,4-diamino-2-methoxyanthraquinone (C. I. Disperse Red 11), 1-amino-2-methylanthraquinone (C. I. Disperse Orange 11), 1-Amino-4-hydroxyanthraquinone (C. I. Disperse Red 15), 1,4,5,8-tetraaminoanthraquinone (C. I. Disperse Blue 1) and 1,4-diamino-5-nitroanthraquinone (C. I. Disperse Violet 8), among others(C.I. Generic Names in parentheses). Aside from the above dyes, the dyes known as C. I. Solvent Blue 14, C. I. Solvent Blue 63, C. I. Solvent Violet 13, C. I. Solvent Violet 14, C. I. Solvent Red 52, C. I. Solvent Red 114, C. I. Vat Blue 21, C. I. Vat Blue 30, C. I. Vat Violet 15, C. I. Vat Violet 17, C. I. Vat Red 19, C. I. Vat Red 28, C. I. Acid Blue 23, C. I. Acid Blue 80, C. I. Acid Violet 43, C. I. Acid Violet 48, C. I. Acid Red 81, C. I. Acid Red 83, C. I. Reactive Blue 4, C. I. Reactive Blue 19, C. I. Disperse Blue 7, etc. can also be employed. These anthraquinone dyes can be used each independently or in a combination of two or more species. Among said anthraquinone dyes, C. I. Disperse Blue 7, C. I. Disperse Violet 1, etc. are preferred. Furthermore, in the present invention, the ozone detection sensitivity can be adjusted by varying the kind (molecular structure etc.) of anthraquinone dye.

The anthraquinone dye content of the ozone sensitive ink can be judiciously selected according to the kind of dye, among other variables, but the usual level of the dye in the ozone sensitive ink may be about 0.05˜10 weight %, preferably 0.05˜5 weight %, more preferably 0.1˜2 weight %. In the present invention, it is more preferable to incorporate a cationic surfactant of the quaternary ammonium salt type in the ink containing said anthraquinone dye.

The cationic surfactant of the quaternary ammonium salt type (hereinafter referred to sometimes briefly as “cationic surfactant”) is not particularly restricted but alkylammonium salts, which may be commercial products, can be employed. These surfactants can be used each independently or in a combination of two or more species. In the practice of the invention, an improved sensitivity of ozone detection can be obtained by using such a cationic surfactant in combination with said anthraquinone dye.

The preferred, among said cationic surfactants, are alkyltrimethylammonium salts and dialkyldimethylammonium salts. Specifically, coco-alkyltrimethylammonium chloride, tallow-alkyltrimethylammonium chloride, behenyltrimethylammonium chloride, hexadecyltrimethylammonium chloride, lauryltrimethylammonium chloride, octadecyltrimethylammonium chloride, dioctyl-dimethylammonium chloride, distearyldimethylammonium chloride, alkylbenzyldimethylammonium chloride, etc. can be mentioned, with lauryltrimethylammonium chloride being particularly preferred.

The level of the cationic surfactant can be judiciously selected according to the kind of surfactant but the usual concentration of the cationic surfactant in the ozone sensitive ink may be about 0.2˜30 weight %, preferably 0.5˜10 weight %.

Where necessary, the ink of the invention may be supplemented with suitable amounts of the known formulating additives for inks, such as a resinous binder, an extender, a solvent and so forth. Moreover, a color component which does not change color in an ozone atmosphere may also be incorporated.

The resinous binder can be judiciously selected according to the type of substrate and, for example, the resin components used in the known ink compositions for writing or printing use can be used as they are. Specifically, maleic acid resins, amide resins, ketone resins, alkylphenol resins, rosin-modified resins, polyvinyl butyral, polyvinylpyrrolidone, cellulosic resins, acrylic resins, vinyl acetate series resins, etc. can be mentioned.

The resinous binder content of the ink can be judiciously selected according to the kind of binder but the usual concentration in the ozone sensitive ink may be not more than 50 weight %, preferably 5˜35 weight %.

The extender is not particularly restricted but includes bentonite, active clay, aluminum oxide and silica gel, among others. Aside from these, the materials known as extender pigments can also be employed. The preferred, among these, are porous materials, with silica gel being particularly preferred. By adding such an extender, chiefly detection sensitivity can be improved.

The level of the extender can be judiciously selected according to the kind of extender but the usual concentration in the ozone sensitive ink may be about 1˜30 weight %, preferably 2˜20 weight %.

The solvent which can be used in this invention includes any and all solvents that are in routine use in the ink compositions for printing or writing. For example, various kinds of solvents inclusive of those in the alcohol series, ester series, ether series, ketone series and hydrocarbon series can be judiciously selected according to the kind of dye to be used, the solubility of the resinous binder, and other variables. The level of the solvent can be adjusted so that, inclusive of the components other than the solvent, the total amount will be 100 weight %. Usually, a suitable level can be liberally established within the range up to 95 weight %.

The color component which does not change color in an ozone atmosphere is not particularly restricted but includes commercial standard color inks, oil-soluble dyes or pigments, and so forth. These may be used each independently or in a combination of two or more species. By formulating said color component, the color change may be made more easily discernible by the eye.

The level of use of said color component can be judiciously set according to the type of color to be used. The usual level in the ozone sensitive ink, however, may be about 0.05˜10 weight %, preferably 0.1˜3 weight %.

The above components may be formulated at one time or in sequence and blended evenly by means of a known mixer such as a homogenizer, a dissolver or the like. For example, a typical procedure comprises formulating the anthraquinone dye, cationic surfactant, resinous binder, extender and so forth into a solvent and mixing the formulation.

Meanwhile, the overcoat layer is constructed on part or the whole of the surface of said color change layer. In the present invention, the overcoat layer is formed preferably on the entire exposed surface of the color-change layer, that is the surface exposed to an ozone atmosphere. Where necessary, two or more such overcoat layers may be provided.

The overcoat layer preferably contains a film-forming polymer. It is particularly preferable that the overcoat layer be substantially exclusively composed of the film-forming polymer. In this arrangement, the color change of the color-change layer can be positively and more easily recognized. Therefore, in the present invention, the overcoat layer preferably does not contain a coloring agent.

In the present invention, a water-soluble polymer can be used with advantage as said film-forming polymer. The kind of polymer is not particularly restricted but can be judiciously selected from among known water-soluble polymers according to the performance and intended application of the ozone indicator. Thus, for example, not only water-soluble resins such as polyvinyl alcohol, polyethylene glycol, polypropylene glycol, polyacrylamide, polyacrylic acid, polyvinylpyrrolidone, water-soluble alkyd resin, polyvinyl ether, polymaleic acid copolymer, polyethyleneimine, etc. but also various starches such as soluble starch, carboxyl-starch, British gum, dialdehyde starch, dextrin, cationic starch, etc.; and cellulosics such as viscose, methylcellulose, ethylcellulose, carboxymethylcellulose, hydroxyethylcellulose, etc. can be mentioned. These can be used each independently or two or more of them may be used together. The particularly preferred, among these, are carboxymethylcellulose, hydroxyethylcellulose, polyvinyl alcohol, polyethylene glycol and polypropylene glycol.

Referring to the film-forming polymers other than water-soluble polymers, there can be mentioned, maleic acid resins, amide resins, ketone resins, alkylphenol resins, rosin-modified resins, polyvinyl butyral, polyvinylpyrrolidone, cellulosic resins, acrylic resins, vinyl acetate resins or the like. These can also be used respectively in the form of an emulsion resin.

The overcoatlayer can be typically formed by using a coating solution prepared by dissolving said film-forming polymer in water or an aqueous solvent inclusive of an alcohol (water is particularly preferred). In this case, the overcoat layer is substantially composed of the water-soluble polymer.

The concentration of the film-forming polymer in the coating solution can be judiciously set according to the kind of film-forming polymer and the desired sensitivity of the ozone indicator, among other variables, but the usual concentration may be about 1˜30 weight %, preferably 5˜25 weight %.

The coating solution may be supplemented with an antifoam and/or other additives within the range not detracting from the effect of the invention.

In the present invention, optionally a non-color-change layer, that is a layer which does not change color even in the presence of ozone, can be provided as well as the color-change layer and overcoat layer. Formation of the non-color-change layer further facilitates recognition of a change of color and provides a greater freedom in design.

The non-color-change layer can be usually formed of an ink which does not change color in the presence of ozone. Moreover, the non-color-change layer includes a substrate insofar as the substrate is one which does not change color in the presence of ozone. Therefore, the artifact comprising a substrate which does not change color in the presence of ozone and, as constructed thereon, a color change layer composed of an ozone sensitive ink can also be used as the ozone indicator according to the present invention. Furthermore, the artifact with a color-change layer formed by saturating a substrate which does not change color in the presence of ozone (e.g. paper, nonwoven cloth or the like) with an ozone sensitive ink can also be used as the ozone indicator according to the invention. In this case, the substrate is the non-color-change layer. The non-color-change layer can be formed in the same manner as the color-change layer.

The ink for use in the formation of a non-color-change layer includes any and all inks so long as the ink or inks are those which do not change color in the presence of ozone. As such inks, commercial regular inks can be used. For example, water-based inks, oil-based inks, solvent-less inks, etc. can be used. In the case of printing, the known relief printing ink, gravure printing ink, screen printing ink, offset printing ink, etc. can be selectively used according to the printing method employed. Those inks can be used each independently or blended for color mixing. It should be understood that the ink for the non-color-change layer may contain those components which are usually included in known inks (e.g. resinous binder, extender, solvent, etc.).

Formation of the color-change layer, non-color-change layer and overcoat layer in the present invention can respectively be carried out by the known printing techniques such as silk screen printing, gravure printing, offset printing, relief printing, flexo printing or the like. Each layer can also be formed by dipping the substrate in an ozone sensitive ink. Ink-permeable materials such as paper and nonwoven cloth are particularly suited. The thicknesses of the color change layer, non-color-change layer and overcoat layer can be judiciously selected according to the type of material for each layer and the intended use of the ozone indicator, among other variables.

In the ozone indicator of the present invention, each of the color-change layer, non-color-change layer and overcoat layer may be provided only as a single layer or as a plurality of layers. A color-change layer may be laminated to another color-change layer. In this case, the color-change layers may be the same or different in composition. A non-color-change layer may be laminated to another non-color-change layer. Then, the non-color-change layers may be the same or different in composition. Similarly, when an overcoat layer is laminated to another overcoat layer, the two layers may be the same or different in composition.

Each layer may be formed over the entire surface of the substrate or another layer or only over part of the surface. In this case, too, it is necessary to construct respective layers in a manner enabling a change of color of the color-change layer.

The particularly preferred is an ozone indicator comprising a substrate and, as disposed thereon, (1) a color-change layer comprised of an ozone sensitive ink, (2) a non-color-change layer, and (3) as formed on part or the whole of the surface of said color-change layer, an overcoat layer, wherein the respective layers are formed in such a manner that part or the whole of said color-change layer may be exposed to ozone to undergo change in color.

The color-change layer may be allowed to undergo change in color by, for example, forming the non-color-change layer on the color-change layer in such a manner that at least a part of the color-change layer will be exposed to an ozone atmosphere and disposing the overcoat layer to cover the exposed part or whole of the color-change layer. In this arrangement, the non-color-change layer may also be covered with the overcoat layer.

Furthermore, the color-change-layer and the non-color-change layer may be formed in such a manner that the color difference between the color-change layer and the non-color-change layer may become recognizable only after the color-change layer has undergone change in color or the respective layers may be formed in such a manner that only color change of the color-change layer will result in no color difference. It is particularly preferable to form the color-change layer and the non-color-change layer in such a manner that the color difference between the color-change layer and the non-color-change layer will become noticeable only after the color-change layer has undergone change in color.

When it is so arranged that a color difference may become recognizable, the color-change layer and the non-color-change layer may for example be formed in such a manner that at least one of a character, a pattern and a symbol will appear on change in color of the color-change layer. In the context of the present invention, said character, pattern and symbol includes all information directing attention to a change in color (presence of ozone). The character and others mentioned above can be designed as desired according to the intended use.

While it may also be arranged that the color-change layer before color change and the non-color change layer are dissimilar in color, it may likewise be so arranged that the color difference (contrast) between the color-change layer and the non-color-change layer will become recognizable only after the former layer has undergone change in color.

In the ozone indicator of the invention, the color-change layer and the non-color-change layer may be formed in such a manner that there will be no overlap of layers. By dosing so, the amount of ink can be economized. By way of illustration, the color-change layer may be formed on part of the substrate surface, while the non-color-change layer may be formed either on the remaining part or the substrate may be left exposed.

Furthermore, in the present invention, a further color-change layer or non-color-change layer may be disposed on at least one of said color-change layer and non-color-change layer. For example, when a color-change layer and a non-color-change layer are formed avoiding an overlap (a color-change/non-color-change layer) and a color-change layer having another design is formed in superimposition, the borderline between the color-change layer and non-color-change layer in said color-change/non-color-change layer can be made substantially unrecognizable so that a more attractive appearance can be imparted.

The ozone indicator of the invention, when disposed in an ozone atmosphere, detects the concentration of ozone and CT value (ozone concentration × exposure time) more accurately and rapidly. Particularly since the ozone indicator of the invention has an overcoat layer, not only qualitative detection but also quantitative detection can be made even in an atmosphere containing a high concentration of ozone. For example, this ozone indicator is applicable to an ozone atmosphere of 1000 ppm or higher ozone concentration, particularly 3000 ppm or higher concentration. Because of these characteristics, the ozone indicator of the invention can be used with advantage in the sterilization or disinfection of foods, medical devices, etc. or in the sterilization, disinfection and deodorizing of atmospheres in operation rooms, clean rooms and so on.

Particularly in the present invention, based on known data about the relationship of CT to color difference (ΔE) and the relationship of CT to the size of color change zone, the ozone concentration and CT value, among others, can be found qualitatively or quantitatively from the actually detected color difference and size of color change zone. For example, by providing a scale or graduations corresponding to CT values along the color-change layer of the ozone indicator of the invention, the CT value can be determined quantitatively according to the size of color-change zone of the color-change layer. Furthermore, based on the CT value found, it is possible to determine the ozone concentration or the exposure time quantitatively or qualitatively.

In addition, not only the CT dependency but also the temperature dependency and humidity dependency of the ozone indicator can be freely controlled. For example, the indicator can be so controlled that the degree of color change will be increased or diminished (or no color change will occur) at a certain temperature or humidity. In other words, the ozone indicator of the invention can be designed so that the degree of color change will vary according to temperature or humidity.

Since the ozone indicator of the invention has a herein-defined overcoat layer, it detects ozone qualitatively and quantitatively even in an atmosphere of high ozone concentration. Particularly, it detects ozone quantitatively even when the ozone concentration is as high as not less than 1000 ppm.

When an anthraquinone dye having at least one amino group species selected from the class consisting of primary and secondary amino groups is used for the color-change layer, the dye reacts effectively with ozone to undergo change in color even under substantially anhydrous conditions, thus exhibiting still higher detection accuracy (selectivity), sensitivity and stability, with the result that the presence of ozone can be easily ascertained even by the naked eye.

Furthermore, the detection sensitivity, speed of color change, etc. can be freely controlled by varying the kinds and formulating ratio of components such as said anthraquinone dye etc. to thereby insure a more sensitive detection of ozone concentration. In addition, through formation of an overcoat layer, the indicator can be so designed that the degree of color change will vary with different temperature or humidity values.

When the ozone sensitive ink of the invention contains a color component which does not change color in an ozone atmosphere, the color change of the ozone-responsive color-change layer formed of this ink can be more easily recognized by the naked eye. Thus, the indicator can be so designed that the color change will be a change from a given color to another color, thus enabling a easier and more accurate visual recognition of color change. As a result, a high ozone detection sensitivity can be obtained and a more accurate quantification of ozone can be made feasible.

### EXAMPLES

The following working and comparative examples illustrate the characteristic feature of the present invention. It should, however, be understood that the invention is made by no means restricted to these examples.

### Example 1

Using a mixer, 0.4 part by weight of anthraquinone disperse dye (“Miketon Fast Red Violet R”, product of Mitsui BASF Co.), 7.5 parts by weight of the resinous binder ethylcellulose resin (“Ethocel 10”, product of Dow Chemical Co.), 2 parts by weight of cationic surfactant coco-alkyltrimethylammonium chloride (“CA-2150”, product of Nikkol Chemical Co.) and 90.1 parts by weight of the solvent ethyl-Cellosolve (“Seefozole MG”, product of Nippon Shokubai Co.) were evenly admixed to prepare an ozone sensitive ink. On the other hand, as the coating solution for construction of an overcoat layer, carboxymethylcellulose (“Cellogen 5A”, product of Daiichi Kogyo Seiyaku Co.) was dissolved in water to prepare a 10 weight % carboxymethylcellulose solution.

On a substrate (Kent paper), the above ozone sensitive ink was silk screen-printed using a 120-mesh silk screen to form a color-change layer on the entire surface of the substrate. Then, on the entire surface of this color-change layer, the above coating solution was silk screen-printed using a 100-mesh silk screen to fabricate an ozone indicator sheet.

### Example 2

Using a mixer, 0.9 part by weight of anthraquinone disperse dye (“Miketon Fast Red Violet R”, product of Mitsui BASF Co.), 7.5 parts by weight of the resinous binder ethylcellulose resin (“Ethocel 10”, product of Dow Chemical Co.), 2 parts by weight of the cationic surfactant coco-alkyltrimethylammonium chloride (“CA-2150”, product of Nikkol Chemical Co.), 10 parts by weight of silica gel (“Aerosil R-972”, product of Japan Aerosil Co.) and 89.6 parts by weight of the solvent ethyl-Cellosolve (Seefozole MG, product of Nippon Shokubai Co.) were evenly admixed to prepare an ozone sensitive ink.

On a substrate (Kent paper), the above ozone sensitive ink was silk screen-printed using a 120-mesh silk screen to construct a color-change layer on the entire surface of the substrate. Then, on the entire surface on this color change layer, the same coating solution as used in Example 1 was silk screen-printed in the same manner as in Example 1 to fabricate an ozone indicator sheet.

### Comparative Example 1

Except that no overcoat layer was constructed, an ozone indicator sheet was fabricated in otherwise the same manner as Example 1.

### Comparative Example 2

Except that no overcoat layer was constructed, an ozone indicator sheet was fabricated in otherwise the same manner as in Example 2.

### Test Example 1

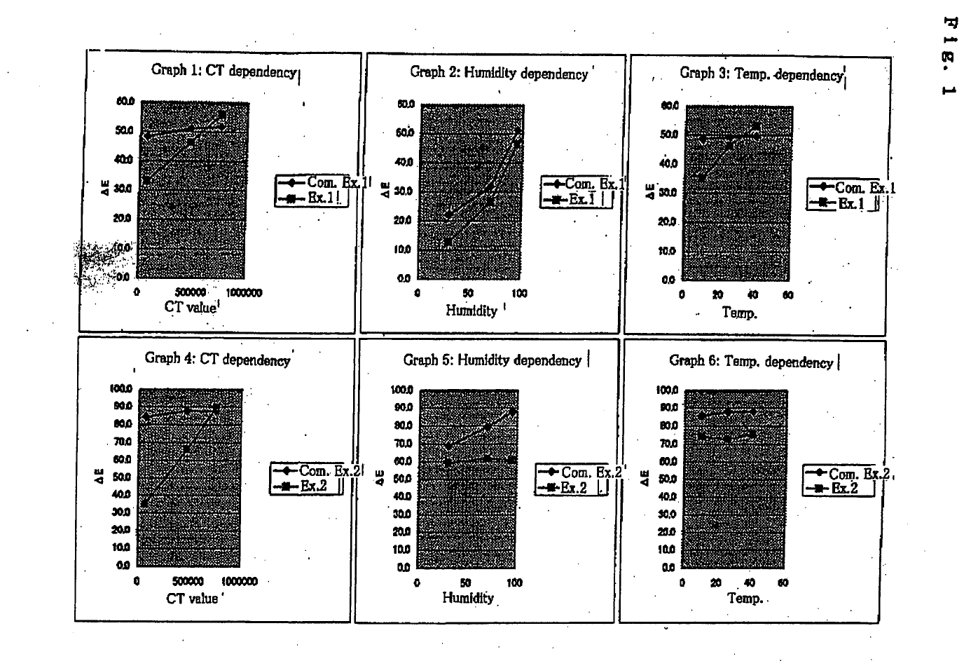

For the ozone indicator fabricated in each the above examples and comparative examples, CT dependency, temperature dependency and humidity dependency were investigated. The results are presented in FIG. 1.

Regarding CT dependency, the color change occurring when the CT value was varied over the range of 70000 to 756000 under the conditions of the temperature 25EC, humidity 95% RH was evaluated in terms of color difference ΔE.

Referring to humidity dependency, the color change occurring when the relative humidity was varied among 30% RH, 70% RH and 95% RH under the conditions of temperature 25EC, CT 470000 was evaluated in terms of color difference ΔE.

As to temperature dependency, the color change occurring when the temperature was varied among 10EC, 25EC and 40EC under the conditions of CT 400000, humidity 95% RH was evaluated in terms of color difference ΔE.

It will be apparent from FIG. 1 that the ozone indicators of the invention exhibit discrete color changes depending on CT, enabling an accurate determination of ozone even at comparatively high ozone concentrations.

It can also be seen that whereas the degree of color change differs according to humidity or temperature in Example 1, there is no variation in the degree of color change in accordance with humidity or temperature in Example 2. Stated differently, in the ozone indicator of the present invention, not only CT dependency but also humidity dependency and temperature dependency can be freely controlled.

Example 3

Using a mixer, 1.68 parts by weight of anthraquinone disperse dye (“Miketon Fast Red Violet R”, product of Mitsui BASF Co.), 0.84 part by weight of oil-soluble dye (“Valifast Yellow 4120”, product of Orient Chemical Industries; C.I. Solvent Yellow 82), 6.55 parts by weight of the resinous binder ethylcellulose resin (“Ethocel 10”, product of DOW Chemical Co.), 7.20 parts by weight of the extender silica gel (“Aerosil R-972”, product of Japan Aerosil Co.), 2.06 parts by weight of coco-alkyltrimethylammonium chloride (“CA-2150”, product of Nikkol Chemical Co.), and 81.66 parts by weight of the solvent ethyl-Cellosolve (“Seefozole MG”, product Nippon Shokubai Co.) were evenly admixed to prepare an ozone sensitive ink. Except that this ozone sensitive ink was used, the procedure of Example 1 was repeated to fabricate an ozone indicator sheet.

### Test Example 2

The ozone indicator sheet fabricated in Example 3 was tested as Test Example 1. As a result, change of the printed color from deep red or rouge to yellow was confirmed. The color difference (ΔE) before and after color change was as high as 45.7.

What is claimed is:

1. An ozone indicator comprising at least (1) a color-change layer comprised of an ozone sensitive ink and (2) an overcoat layer formed on part or the whole of the surface of said color-change layer, wherein the ozone sensitive ink comprises a color component which does not change color in an ozone atmosphere.

2. The ozone indicator according to claim 1, wherein the overcoat layer comprises a film-forming polymer.

3. The ozone indicator according to claim 2, wherein the film-forming polymer is selected from the group consisting of carboxymethylcellulose, hydroxyethylcellulose, polyvinyl alcohol, polyethylene glycol and polypropylene glycol.

4. An ozone indicator comprising at least (1) a color-change layer comprised of an ozone sensitive ink, (2) a color-unchanged layer comprised of a color component which does not change color in an ozone atmosphere, and (3) an overcoat layer formed on part of or the whole of the surface of said color-change layer, wherein the color-change layer and the color-unchanged layer are configured to show the color-unchanged layer when color of the color-change layer changes.

5. The ozone indicator according to claim 4, wherein the color-unchanged layer is formed in a character, a pattern, and a symbol.

6. The ozone indicator according to claim 4, wherein the overcoat layer comprises a film-forming polymer.

7. The ozone indicator according to claim 6, wherein the film-forming polymer is selected from the group consisting of carboxymethylcellulose, hydroxyethylcellulose, polyvinyl alcohol, polyethylene glycol and polypropylene glycol.
